(19) United States
(12) Patent Application Publication
(10) Pub. No.: US 2006/0106230 A1
(43) Pub. Date: May 18, 2006

(54) Processes for preparing amorphous atorvastatin hemi-calcium

(76) Inventors: Michael Pinchasov, Rishon-Lezion (IL); Dalia Maidan-Hanoch, Kfar Yona (IL)

Correspondence Address: KENYON & KENYON LLP, ONE BROADWAY, NEW YORK, NY, 10004, US

(21) Appl. No.: 11/253,770

(22) Filed: Oct. 18, 2005

Related U.S. Application Data
(60) Provisional application No. 60/620,022, filed on Oct. 18, 2004.

(51) Int. Cl. C07D 207/14

(52) U.S. Cl. 548/537

(57) ABSTRACT

Novel processes for the preparation of amorphous atorvastatin hemi-calcium salt are provided, which involve dissolving atorvastatin hemi-calcium salt in certain organic solvents, and removing the solvent such as by spray drying, rapid vacuum evaporation, and/or thin film evaporation. Preferred embodiments of these processes for preparing amorphous atorvastatin hemi-calcium salt are reproducible, applicable on a large scale, and do not involve the use of hydrocarbons.

This application claims the benefit of U.S. Provisional Patent Application Ser. No. 60/620,022 filed Oct. 18, 2004, which is incorporated herein by reference.

### FIELD OF THE INVENTION

The present invention is directed to novel processes for the preparation of amorphous atorvastatin and the amorphous atorvastatin material produced by this process, as well as pharmaceutical compositions and methods of treatment using such material.

### BACKGROUND OF THE INVENTION

Atorvastatin, ([R-(R*,R*)]-2-(4-fluorophenyl)-β,δ-dihydroxy-5-(1-methylethyl)-3 phenyl-4-[(phenylamino)carbonyl]-1H-pyrrole-1-heptanoic acid), depicted in lactone form in Formula (I) and its calcium salt trihydrate of Formula (II) (water molecules not shown) are well known in the art, and described, inter alia, in U.S. Pat. Nos. 4,681,893; 5,273,995, and in U.S. Ser. No. 60/166,153, filed Nov. 17, 2000, all of which are herein incorporated by reference.

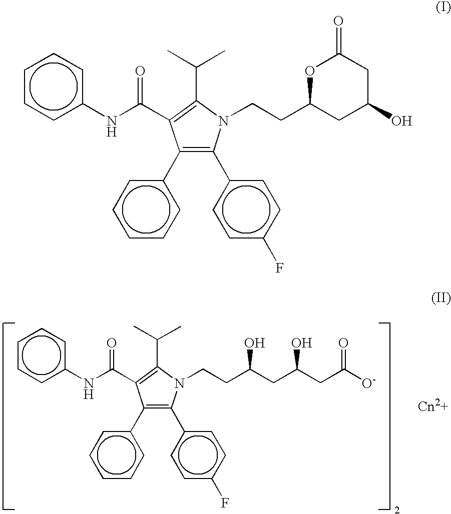

Atorvastatin is a member of the class of drugs called statins. Statin drugs are currently the most therapeutically effective drugs available for reducing low density lipoprotein (LDL) particle concentration in the blood stream of patients at risk for cardiovascular disease. A high level of LDL in the bloodstream has been linked to the formation of coronary lesions which obstruct the flow of blood and can rupture and promote thrombosis. Goodman and Gilman, The Pharmacological Basis of Therapeutics 879 (9th ed. 1996). Reducing plasma LDL levels has been shown to reduce the risk of clinical events in patients with cardiovascular disease and patients who are free of cardiovascular disease but who have hypercholesterolemia. Scandinavian Simvastatin Survival Study Group, 1994; Lipid Research Clinics Program, 1984a, 1984b.

The mechanism of action of statin drugs has been elucidated in some detail. They interfere with the synthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion of HMG to mevalonate, which is the rate determining step in the biosynthesis of cholesterol, and so, its inhibition leads to a reduction in the concentration of cholesterol in the liver. Very low density lipoprotein (VLDL) is the biological vehicle for transporting cholesterol and triglycerides from the liver to peripheral cells. VLDL is catabolized in the peripheral cells which release fatty acids which may be stored in adipocytes or oxidized by muscle. The VLDL is converted to intermediate density lipoprotein (IDL), which is either removed by an LDL receptor, or is converted to LDL. Decreased production of cholesterol leads to an increase in the number of LDL receptors and corresponding reduction in the production of LDL particles by metabolism of IDL.

Atorvastatin was first disclosed to the public and claimed in U.S. Pat. No. 4,681,893. The hemi-calcium salt is disclosed in U.S. Pat. No. 5,273,995. The '995 patent teaches that the hemi-calcium salt is obtained by crystallization from a brine solution resulting from the transposition of the sodium salt with CaCl₂ and further purified by recrystallization from a 5:3 mixture of ethyl acetate and hexane. Atorvastatin calcium is marketed under the name LIPITOR® by Pfizer, Inc.

Distinct crystalline forms are disclosed in several patents and patent applications. Crystalline Forms I, II, III and IV of atorvastatin calcium are the subjects of U.S. Pat. Nos. 5,969,156 and 6,121,461 assigned to Warner-Lambert. Polymorph forms of atorvastatin designated Forms V, VI, VIII, IX, X, XI and XII and novel processes for their preparation are described in commonly-owned published application nos. WO 01/36384 and US 2002/0183378, which are herein incorporated by reference.

WO 03/099785 discloses a process for the preparation of amorphous atorvastatin calcium comprising dissolving Form I or a mixture of crystalline and amorphous atorvastatin calcium in a solvent consisting of an aliphatic acyclic ketone, filtering the solution and removing the solvent at 40 to 50° C. under vacuum.

WO 03/093233 discloses a process for the preparation of amorphous atorvastatin calcium comprising dissolving atorvastatin calcium salt in an organic solvent miscible with water, gradually adding said solution to water while stirring, and filtering and vacuum drying the solid obtained.

WO 03/068739 discloses a process for the preparation of amorphous atorvastatin calcium comprising forming the hemi-calcium salt of atorvastatin in solution and precipitating amorphous atorvastatin from the solution with a C₅-C₁₂ hydrocarbon (e.g., hexane) or a dialkyl ether.

WO 03/078379 discloses a process for the preparation of amorphous atorvastatin calcium comprising dissolving a mixture of amorphous and crystalline atorvastatin in a hydroxylic solvent followed by freeze drying or spray drying.

U.S. Pat. No. 6,613,916 discloses a process for the preparation of amorphous atorvastatin calcium comprising dissolving atorvastatin in a solvent in which it is freely soluble, such as a low molecular weight alcohol, ketone, or ester and precipitating amorphous atorvastatin with a solvent in which atorvastatin is insoluble or only very slightly soluble, such as an ether.

U.S. Pat. No. 6,646,133 discloses a process for the preparation of amorphous atorvastatin calcium comprising dissolving crude amorphous atorvastatin calcium in a lower alkanol containing 2-4 carbon atoms or a mixture of such alkanols under heating and isolating the amorphous atorvastatin calcium precipitated after cooling.

U.S. Pat. No. 6,274,740 discloses a process for the preparation of amorphous atorvastatin calcium comprising dissolving atorvastatin in tetrahydrofuran or a mixture of tetrahydrofuran and toluene.

U.S. Pat. No. 6,528,660 discloses a process for the preparation of amorphous atorvastatin calcium and hydrates thereof which comprises: (a) dissolving crystalline atorvastatin calcium in a non-hydroxylic solvent; (b) adding a non-polar hydrocarbon anti-solvent or adding the dissolved atorvastatin to the non-polar anti-solvent to precipitate out atorvastatin calcium; and (c) removing the solvent by filtration to afford amorphous atorvastatin calcium.

WO 02/43732 discloses processes for the preparation of amorphous atorvastatin calcium comprising treating any other form of atorvastatin hemi-calcium with acetone at room temperature to reflux temperature for between a few hours and 25 hours, followed by drying in a drying oven, or by sonicating any form of atorvastatin hemi-calcium in acetonitrile at any temperature between room temperature and the reflux temperature of acetonitrile. Also disclosed is a process for preparing amorphous atorvastatin hemi-calcium by ball milling of any crystalline form of atorvastatin hemi-calcium.

WO 02/057228 discloses a process for the preparation of amorphous atorvastatin calcium comprising dissolving a mixture of amorphous and crystalline atorvastatin in a non-hydroxylic solvent followed by the addition of a suitable non-hydroxylic solvent to precipitate the product which is then isolated. Alternatively, the solution of atorvastatin in a non-hydroxylic solvent is added to a non-hydroxylic solvent to induce precipitation. The product can be isolated by such methods as filtration, centrifugation or decantation.

WO 02/059087 discloses a process for the preparation of amorphous atorvastatin hemi-calcium salt in which atorvastatin in the form of either the lactone or a hydroxy and carboxylic acid protected form in a non-hydroxylic solvent is converted to the free hydroxy, free acid and then treated with water followed by a solvent immiscible or slightly miscible with water.

WO 02/083637 discloses a process for the preparation of amorphous atorvastatin hemi-calcium salt comprising i) treating diol protected tert-butyl ester with a methanolic solution in the presence of an aqueous acid; (ii) adding aqueous hydroxide solution to the reaction mixture; and removing unreacted diol protected tert-butyl ester by solvent extraction (iii) treating the product obtained in step (ii) with calcium chloride solution to obtain crude amorphous atorvastatin calcium salt; (iv) isolating said crude salt; (v) treating the crude salt with excess volume of methanol; (vi) treating the product of step (v) with activated carbon and (vii) precipitation of the product by adding methanolic solution of atorvastatin calcium in to water (viii) recovering the pure product by filtration and drying.

WO 02/083638 discloses a process for the preparation of amorphous atorvastatin hemi-calcium salt comprising (i) treating diol protected tert-butyl ester with a methanolic solution in the presence of an aqueous acid; (ii) adding aqueous hydroxide solution to the reaction mixture; and removing unreacted diol protected tert-butyl ester by solvent extraction (iii) treating the product obtained in step (ii) with calcium chloride solution to obtain crude amorphous atorvastatin calcium salt; (iv) isolating said crude salt; (v) treating crude product so isolated with activated carbon in aqueous ethyl acetate (vi) recovering the product by addition of non polar hydrocarbon solvent, followed by filtration and drying to produce amorphous atorvastatin calcium.

WO 03/018547 discloses a process for the preparation of amorphous atorvastatin hemi-calcium salt comprising hydrolyzing the lactone form of atorvastatin with aqueous alkali or alkaline earth metal base, extracting with organic solvent the reaction mixture, adding the same to an anti-solvent to precipitate the product, and finally filtering the product

WO 2004/043918 discloses processes for the preparation of amorphous atorvastatin calcium comprising dissolving atorvastatin hemi-calcium in acetone at room temperature to reflux temperature, sonicating atorvastatin hemi-calcium in acetonitrile, ball milling of any crystalline form of atorvastatin hemi-calcium, and dissolving atovastatin in a mixture of 1-butanol and water.

Prior art methods of preparing amorphous atorvastatin after dissolving atorvastatin in organic solvents such as, e.g., ketones, generally involved the step of drying the product in an oven at elevated temperatures for prolonged times. For example, WO 01/28999 discloses a process that involves dissolving atorvastatin in an aliphatic acyclic ketone followed by drying the product under vacuum at 45° C. to 50° C. for 10 to 15 hours. Such methods run the risk of causing appreciable degradation of the product due to the long exposure to elevated temperatures. In contrast, the present invention, involves drying steps that are carried out by spray drying, rapid vacuum evaporation, or thin film evaporation. Surprisingly, these more gentle methods preferably provide a product with less or no degradation products, and within the permitted limits for residual solvent The present methods thus represent a considerable advance over the prior art.

While some prior art discloses the preparation of amorphous atorvastatin from a solution of atorvastatin in ethyl acetate, such prior art generally requires the use of non-polar solvents such as hexane to precipitate the amorphous atorvastatin (see, e.g., WO 03/068739). This leads to the presence of contaminating amounts of the non-polar solvent in the product, an undesirable outcome. Preferred embodiments of the present invention avoid the use of such unfavorable solvents.

### SUMMARY OF THE INVENTION

In one embodiment, the present invention provides processes for preparing amorphous atorvastatin hemi-calcium salt comprising the steps of: dissolving atorvastatin hemi-calcium salt in an organic solvent that is a mixture of hydroxylic solvent with a solvent selected from ketone, ester or a mixture thereof, removing the organic solvent from the solution and recovering the amorphous form.

In another embodiment, the present invention provides processes for preparing amorphous atorvastatin hemi-calcium salt comprising the steps of: dissolving atorvastatin hemi-calcium salt in an organic solvent selected from the group consisting of: ketones, esters and mixtures thereof, preferably in a concentration of less than about 25% or more than about 40%, removing the organic solvent from the solution and recovering the amorphous form.

In yet another embodiment, the present invention provides processes for preparing amorphous atorvastatin hemi-calcium salt comprising the steps of: dissolving crystalline form selected from the group consisting of: V, VI, VII, XI, XII, XV, XVIII, and XIX of atorvastatin hemi-calcium salt in an organic solvent selected from the group consisting of: ketones, esters, mixtures thereof or their mixture with hydroxylic solvents, removing the organic solvent from the solution and recovering the amorphous form.

The solvent is removed by using drying technology such as, for example, spray drying, thin film evaporation, or rapid vacuum evaporation to obtain amorphous atorvastatin hemi-calcium salt. The steps of spray drying, thin film evaporation, and rapid vacuum drying can be carried out by methods known in the art.

### DETAILED DESCRIPTION

As used herein, “amorphous” means a solid with no substantial long range crystalline order. Amorphous atorvastatin hemi-calcium salt in accordance with this application preferably contains less than about 10% crystalline atorvastatin hemi-calcium salt, and more preferably is essentially free of crystalline atorvastatin hemi-calcium salt.

As used herein, “Essentially free of crystalline atorvastatin hemi-calcium salt” means that no crystalline atorvastatin hemi-calcium salt can be detected within the limits of powder X-ray diffraction, for example.

As used herein, the term “ester”, unless otherwise stated, refers to a compound of formula RₐCO₂R_{b} wherein Rₐ and R_{b} are C₁₋₆ alkyl radicals which may be the same or different.

As used herein, the term “ketone”, unless otherwise stated, refers to a compound of formula RₐCOR_{b} wherein Rₐ and R_{b} are C₁₋₆ alkyl radicals which may be the same or different.

As used herein, the term “hydroxylic solvent”, unless otherwise stated, refers to a compound of formula ROH wherein R is C₁₋₆ alkyl radicals (aliphatic or branched).

The present invention provides processes for preparing amorphous atorvastatin hemi-calcium salt that are reproducible, applicable on a large scale, and that advantageously do not involve the use of hydrocarbons.

In one embodiment, the present invention provides processes for preparing amorphous atorvastatin hemi-calcium salt comprising the steps of: dissolving atorvastatin hemi-calcium salt in an organic solvent that is a mixture of hydroxylic solvent with a solvent selected from ketone, ester or a mixture thereof, removing the organic solvent from the solution and recovering the amorphous form.

Preferably, the hydroxylic solvent is a C₁-C₄ alcohol. More preferably, the hydroxylic solvent is methanol, ethanol, n-propanol, isopropanol, n-butanol, isobutanol, or t-butanol. In certain embodiments, the hydroxylic solvent is not methanol.

The use of hydroxylic solvents in a mixture with ketones and/or esters, as described above, results in an azeotropic mixture with a lower boiling point in comparison to the hydroxylic solvent alone. For example, the boiling point of methanol is 65° C., while in a mixture with acetone the boiling point is 55° C. The boiling point of ethyl acetate is 77° C., while in a mixture with methanol the boiling point is 62° C. High temperature may cause the formation of degradation products in atorvastatin hemi-calcium salt. Thus, lowering the boiling point of the reaction mixture may prevent the formation of such degradation products.

In another embodiment, the present invention provides processes for preparing amorphous atorvastatin hemi-calcium salt comprising the steps of: dissolving atorvastatin hemi-calcium salt in an organic solvent selected from the group consisting of: ketones, esters and mixtures thereof, preferably in a concentration of less than about 25% or more than about 40%, removing the organic solvent from the solution and recovering the amorphous form.

Ketones and esters in a concentration of more than about 40% form atorvastatin hemi-calcium having bigger particles that reduce the mechanical lost of the obtained product, and eventually results a higher yield. Ketones and esters in a concentration of less than about 25%, requires lower temperature in order to dissolve the material, and as a result the exposure to heat is lower, and the product decomposes less.

In another embodiment, the present invention provides processes for preparing amorphous atorvastatin hemi-calcium salt comprising the steps of: dissolving crystalline form selected from the group consisting of: V, VI, VII, XI, XII, XV, XVIII, and XIX of atorvastatin hemi-calcium salt in an organic solvent selected from the group consisting of: ketones, esters, mixtures thereof or their mixture with hydroxylic solvents, removing the organic solvent from the solution and recovering the amorphous form. The preferred polymorphic forms have a lower crystallinity in comparison to Form I, which is described in the prior art, and as a result, they are more soluble in non-hydroxylic solvents such as ketones and esters.

Preferably, the dissolving step comprises heating the mixture of atorvastatin hemi-calcium salt in the organic solvent. More preferably, the heating is at a temperature of about 40° C. to about 100° C., until obtaining complete dissolution. Most preferably, the heating is at a temperature of about 80° C.

The atorvastatin calcium may be any calcium salt of atorvastatin, including but not limited to atorvastatin hemi-calcium (2:1).

Preferably, the ketone is selected from the group consisting of acetone, methylethyl ketone, methylbutyl ketone and combinations thereof.

Preferably, the ester is selected from the group consisting of ethyl acetate, methyl acetate, isobutyl acetate, and combinations thereof.

Preferably, the ratio of atorvastatin hemi-calcium salt to the organic solvent is about 1 g/1 mL to about 1 g/10 mL. More preferably, the ratio is about 1 g/5 mL to about 1 g/10 mL. This range of atorvastatin/solvent ratios is preferable for the specific working temperature range of about 40° C. to about 100° C. Reducing the amount of solvent generally requires the use of higher temperature, and this is undesirable because the stability of the product may be compromised. The dissolving step is preferably carried out at a temperature of about 40° C. to about 100° C. but may be carried out at other temperatures, providing the atorvastatin hemi-calcium salt dissolves sufficiently in the chosen solvent. Routine experimentation will provide suitable ranges of temperature and atorvastatin/solvent ratios.

Preferably, when the organic solvent is hydroxylic solvent the ratio of atorvastatin hemi-calcium to hydroxylic solvent (on a weight/volume basis) is about 1 g/60 ml when dissolving wet material and about 1 g/10 ml when dissolving dry material.

Preferably, when the organic solvent is ethyl acetate, and the ratio of atorvastatin hemi-calcium salt to the organic solvent is about 1 g/5 mL or about 1 g/1 g.

Preferably, when the organic solvent is acetone, and the ratio of atorvastatin hemi-calcium salt to the organic solvent is about 1 g/10 mL or about 1 g/1 g.

Preferably, when the organic solvent is a mixture of an ester and a hydroxylic solvent, the ratio of ester to hydroxylic solvent in the mixture is preferably (on a volume/volume basis) about 1:1.

Preferably, when the organic solvent is a mixture of a ketone and a hydroxylic solvent, the ratio of ketone to hydroxylic solvent in the mixture is preferably (on a volume/volume basis) about 1:1.

The solvent may be removed, for example, by spray drying, thin film evaporation, or rapid vacuum evaporation to obtain amorphous atorvastatin hemi-calcium salt. The steps of spray drying, thin film evaporation, and rapid vacuum drying can be carried out by methods known in the art.

The term “spray drying” broadly refers to processes involving breaking up liquid mixtures into small droplets (atomization) and rapidly removing solvent from the mixture.

In a typical spray drying apparatus, there is a strong driving force for evaporation of solvent from the droplets, which can be provided by providing a drying gas. Spray drying processes and equipment are described in Perry's Chemical Engineer's Handbook, pp. 20-54 to 20-57 (6th ed. 1984).

By way of non-limiting example only, the typical spray drying apparatus includes a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2, and PSD-4 (Niro A/S, Soeborg, Denmark). Commercial equipment for spray drying may be used, such as model AGM-2M-SD by the manufacturer Hosokawa Micron Corporation.

Typically, the product collection means includes a cyclone connected to the drying apparatus. In the cyclone, the particles produced during spray drying are separated from the drying gas and evaporated solvent, allowing the particles to be collected. A filter can also be used to separate and collect the particles produced by spray drying. The process of the invention is not limited to the use of such drying apparatuses as described above.

Spray drying can be performed in a conventional manner in the processes of the present invention. See, e.g., Remington: The Science and Practice of Pharmacy, vol. II, p. 1627 (19th ed. 1995), incorporated herein by reference. The drying gas used in the invention can be any suitable gas, preferably an inert gas such as nitrogen, nitrogen-enriched air, or argon. Nitrogen gas is a particularly preferred drying gas for use in the process of the invention.

Thin film evaporation can be carried out by methods known in the art. A preferred thin film evaporator is the Artisan Rototherm E® Thin Film Evaporator by the manufacturer Artisan Industries Inc. The Artisan Rototherm is a horizontal, mechanically-aided, thin film evaporator. The Artisan Rototherm allows 99%+evaporation in a single pass, concentrating dilute solutions to powder if necessary.

Short reaction time results in superior quality and higher yields when compared to batch processing. In particular, a horizontal, straight-sided design offers certain advantages over other thin film or wiped film evaporated, such as no dry spots or fouling, process wall fully wetted at all evaporation rates and feed rates, thin film unaffected by gravity, etc.

Rapid vacuum evaporation may be carried out by methods known in the art. Any standard method for “rapid vacuum evaporation” may be used. For example, in a typical laboratory vacuum rapid evaporation technique, a solution is fed drop-wise into a pre-heated reactor under vacuum. The product is then unloaded as a dry powder.

The term “rapid vacuum evaporation” includes techniques where the solvent is evaporated under reduced pressure to achieve quick evaporation of the solvent. For example, common laboratory vacuum evaporation techniques include but are not limited to centrifugal evaporation, rotary evaporation, and vortex evaporation.

The rapid vacuum drying step is carried out at suitable times, temperatures, and pressures, depending on the amount of atorvastatin treated. For example, for about 10 g of atorvastatin, the following conditions have been found to be suitable: 15-20 minutes, at a temperature of about 60±5° C. and pressure of 30 mmHg. The solution is fed into the dryer at room temperature.

Preferably, heat is supplied to the sample either from the evaporation chamber/vessel walls or from another external heat source, for example, an infrared heater. The pressure controls the temperature at which the solvent boils. Rapid evaporation of non-volatile solvents is possible at relative high pressures but this can only occur if the samples are relatively hot. The pressure at the sample must be below the vapor pressure of the solvent for boiling to occur and provide rapid evaporation. In most drug discovery applications the sample temperature must be kept below a temperature that would cause heat damage to samples. To achieve this level in the chamber in the vicinity of the samples requires careful design of the vacuum system, including the condenser or cold trap and vacuum connections.

Heat can be applied in a variety of ways. Evaporation normally takes place at pressures which are nearly a complete vacuum in order to keep the sample temperature low. Conduction and convection through the atmosphere in the chamber can only convey very low amounts of heat under these conditions. Radiation from the walls of the chamber also provides very little heat because they are not normally warmed above 40° C. If they are heated above this level, samples could be overheated when dry. Radiation from high temperature infrared sources such as filament lamps can provide much higher heat inputs and because the heated mass is small they can be switched off quickly when the samples are dry so that overheating can be avoided.

Vacuum evaporation is preferably performed at a pressure in the range of from about 3 Torr to about 250 Torr, and at temperatures in the range of from about 20° C. to about 60° C.

The invention also relates to amorphous atorvastatin hemi-calcium salt made by the processes described herein, as well as pharmaceutical compositions and methods for treating hypertension. The amorphous form made by a process described herein preferably has better solubility and flowability and is polymorphically stable.

Amorphous atorvastatin calcium prepared according to the present invention may be characterized by its X-ray powder diffraction pattern. X-ray powder diffraction of amorphous atorvastatin preferably shows no peaks characteristic of crystal forms of atorvastatin, thus demonstrating the amorphous nature of the product. Appropriate X-ray powder diffraction of amorphous atorvastatin is described in the prior art.

Solid amorphous atorvastatin hemi-calcium salts, and solvates thereof can be formulated into a variety of compositions for administration to humans and mammals.

Pharmaceutical compositions of the present invention contain solid amorphous atorvastatin hemi-calcium salt, prodrugs, and/or solvates thereof, optionally in mixture with other crystalline forms and/or other active ingredients. In addition to the active ingredient(s), the pharmaceutical compositions of the present invention can contain one or more excipients.

Solid amorphous atorvastatin hemi-calcium salts and solvates thereof can be administered for treatment of hypertension by any means that delivers the active pharmaceutical ingredient(s) to the site of the body where competitive inhibition of the 3-hydroxy-3-methyl-glutaryl-coenzyme A reductase enzyme (“HMG-CoA reductase”) exerts a therapeutic effect on the patient. For example, administration can be oral, buccal, parenteral (including subcutaneous, intramuscular, and intravenous) rectal, inhalant and ophthalmic. Although the most suitable route in any given case will depend on the nature and severity of the condition being treated, the most preferred route of the present invention is oral. Solid amorphous atorvastatin hemi-calcium salts, and/or solvates thereof can be conveniently administered to a patient in oral unit dosage form and prepared by any of the methods well-known in the pharmaceutical arts. Dosage forms include solid dosage forms like tablets, powders, capsules, sachets, troches and lozenges as well as liquid syrups, suspensions and elixirs. The solid compositions of the present invention include powders, granulates, aggregates and compacted compositions. The active ingredient(s) and excipients can be formulated into compositions and dosage forms according to methods known in the art.

Excipients are added to the composition for a variety of purposes. Examples include diluents, binders, disintegrants, glidants, lubricants, flavorants, dyes, etc. Selection of excipients and the amounts to use can be readily determined by the formulation scientist based upon experience and consideration of standard procedures and reference works in the field.

A composition for tableting or capsule filling can be prepared by methods known in the art, such as wet granulation, dry granulation, direct compression, etc. A capsule filling of the present invention can comprise any of the aforementioned blends and granulates that were described with reference to tableting, only they are not subjected to a final tableting step.

In liquid pharmaceutical compositions of the present invention, solid amorphous atorvastatin hemi-calcium salts, and solvates thereof and any other solid excipients are dissolved or suspended in a liquid carrier such as water, vegetable oil, alcohol, polyethylene glycol, propylene glycol or glycerin.

Liquid pharmaceutical compositions may contain, for example, emulsifying agents, viscosity-enhancing agents, sweetening agents, preservatives, buffers, and/or chelating agents.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

Other suitable formulations for use in the present invention can be found, for example, in Remington's Pharmaceutical Sciences, Mace Publishing Company, Philadelphia, Pa., 17th ed. (1985).

The amount of compound administered to the patient will vary depending upon what is being administered, the purpose of the administration, such as prophylaxis or therapy, the state of the patient, the manner of administration, and the like. In therapeutic applications, compositions are administered to a patient already suffering from hyperlipidemia and/or hypercholesterolemia in an amount sufficient to at least partially arrest further onset of the symptoms of the disease and its complications. An amount adequate to accomplish this is defined as “therapeutically effective dose.” Amounts effective for this use will depend on the judgment of the attending clinician depending upon factors such as the degree or severity of hyperlipidemia and/or hypercholesterolemia in the patient, the age, weight and general condition of the patient, and the like. Capsules, tablets and lozenges and other unit dosage forms preferably contain a dosage level of about 10 mg to about 100 mg, more preferably from about 25 mg to about 50 mg of the amorphous atorvastatin hemi-calcium salt.

In prophylactic applications, compositions are administered to a patient at risk of developing cardiovascular disease (determined for example by screening, familial trait, etc.) in an amount sufficient to inhibit the onset of symptoms of the disease. An amount adequate to accomplish this is defined as “prophylactically effective dose.” Amounts effective for this use will depend on the judgment of the attending clinician depending upon factors such as the age, weight and general condition of the patient, and the like.

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

As noted above, the compounds administered to a patient are in the form of pharmaceutical compositions described above. These compositions may be sterilized by conventional sterilization techniques, or may be sterile filtered. When aqueous solutions are employed, these may be packaged for use as is, or lyophilized, the lyophilized preparation being combined with a sterile aqueous carrier prior to administration.

The following examples are offered to illustrate this invention and are not to be construed in any way as limiting the scope of this invention.

### EXAMPLES

### Example 1

60 g of atorvastatin hemi-calcium salt Form V was dissolved in acetone (600 ml) at 40° C. The solution was spray dried at 60° C. for 18 minutes to obtain amorphous atorvastatin hemi-calcium salt.

### Example 2

59 g of atorvastatin hemi-calcium salt Form V was dissolved in ethyl acetate (295 g) at 60° C. The solution was spray dried at 60° C. for 5 minutes to obtain amorphous atorvastatin hemi-calcium salt.

### Example 3

15 g of wet atorvastatin hemi-calcium salt Form V was dissolved in MeOH (340 ml) at 50° C. The solution was fed into a pre-heated (60° C.) 1 L reactor under vacuum (˜80 mbar) to obtain amorphous atorvastatin hemi-calcium salt. Drying was carried out for about 4 hours.

### Example 4

20 g of atorvastatin hemi-calcium salt Form V was dissolved in acetone (20 g) at about 50° C. The solution was spray to obtain amorphous atorvastatin hemi-calcium salt. The nitrogen gas was at an inlet temperature of 150° C. The evaporated solvent and nitrogen left the spray dryer at a temperature of 95-97° C.

### Example 5

20 g of atorvastatin hemi-calcium salt Form V was dissolved in acetone (80 g) at about 50° C. The solution was spray to obtain amorphous atorvastatin hemi-calcium salt. The nitrogen gas was at an inlet temperature of 150° C. The evaporated solvent and nitrogen left the spray dryer at a temperature of 99° C.

### Example 6

152 g of atorvastatin hemi-calcium salt Form V was dissolved in acetone (798 g) at about 50° C. The solution was spray to obtain amorphous atorvastatin hemi-calcium salt. The nitrogen gas was at an inlet temperature of 150° C. The evaporated solvent and nitrogen left the spray dryer at a temperature of 94-107° C.

### Example 7

20 g of atorvastatin hemi-calcium salt Form V was dissolved in ethyl acetate (80 g) at about 75° C. The solution was spray to obtain amorphous atorvastatin hemi-calcium salt. The nitrogen gas was at an inlet temperature of 100° C. The evaporated solvent and nitrogen left the spray dryer at a temperature of 71-73° C.

### Example 8

50 g of atorvastatin hemi-calcium salt Form V was dissolved in ethyl acetate (50 g) at about 75° C. The solution was spray to obtain amorphous atorvastatin hemi-calcium salt. The nitrogen gas was at an inlet temperature of 150° C. The evaporated solvent and nitrogen left the spray dryer at a temperature of 98-101° C.

Although the above examples have been illustrated with the use of atorvastatin hemi-calcium Form V as starting material, any crystalline form of atorvastatin which dissolves in the chosen solvent may be used instead of atorvastatin Form V.

From the foregoing description, various modifications and changes in the composition and method will occur to those skilled in the art. All such modifications coming within the scope of the appended claims are intended to be included therein.

What is claimed is:

1. A method for preparing amorphous atorvastatin hemi-calcium salt comprising:
a) dissolving atorvastatin hemi-calcium salt in an organic solvent that is a mixture of a first hydroxylic solvent with a second solvent selected from ketone, ester or a mixture thereof; and
b) removing the organic solvent to obtain amorphous atorvastatin hemi-calcium salt.

2. The method of claim 1, wherein the hydroxylic solvent is a C₁-C₄ alcohol

3. The method of claim 2, wherein the hydroxylic solvent is methanol.

4. The method of claim 1, wherein the second solvent is selected from the group consisting of acetone, methylethyl ketone, methylbutyl ketone and combinations thereof.

5. The method claim 4, wherein the second solvent is acetone.

6. The method claim 1, wherein the second solvent is selected from the group consisting of ethyl acetate, methyl acetate, isobutyl acetate, and combinations thereof.

7. The method claim 6, wherein the second solvent is ethyl acetate.

8. A method for preparing amorphous atorvastatin hemi-calcium salt comprising:
a) dissolving atorvastatin hemi-calcium salt in a an organic solvent selected from the group consisting of ketones, esters and mixtures thereof, in a concentration of less than about 25% or more than about 40%; and
b) removing the solvent to obtain amorphous atorvastatin hemi-calcium salt.

9. The method of claim 8, wherein the organic solvent is selected from the group consisting of acetone, methylethyl ketone, methylbutyl ketone and combinations thereof

10. The method claim 9, wherein the organic solvent is acetone.

11. The method claim 8, wherein the organic solvent is selected from the group consisting of ethyl acetate, methyl acetate, isobutyl acetate, and combinations thereof.

12. The method claim 11, wherein the organic solvent is ethyl acetate.

13. A method for preparing amorphous atorvastatin hemi-calcium salt comprising:
a) dissolving crystalline form selected from the group consisting of: V, VI, VII, XI, XII, XV, XVIII, and XIX of atorvastatin hemi-calcium salt in an organic solvent selected from the group consisting of ketones, esters, mixtures thereof or their mixture with hydroxylic solvents; and
b) removing the organic solvent to obtain amorphous atorvastatin hemi-calcium salt.

14. The method of claim 13, wherein the organic solvent is selected from the group consisting of acetone, methylethyl ketone, methylbutyl ketone and combinations thereof.

15. The method of claim 14, wherein the organic solvent is acetone.

16. The method claim 13, wherein the organic solvent is selected from the group consisting of ethyl acetate, methyl acetate, isobutyl acetate, and combinations thereof

17. The method of claim 16, wherein the organic solvent is ethyl acetate.

18. The method of any one of claims 1, 8 and 13 wherein the dissolving step comprises heating the mixture of atorvastatin hemi-calcium salt in the organic solvent.

19. The method of claim 18, wherein the dissolving step is carried out at a temperature of about 40° C. to about 100° C.

20. The method of claim 19, wherein the dissolving step is carried out at a temperature of about 80° C.

21. The method of any one of claims 1, 8 and 13, wherein the ratio of atorvastatin hemi-calcium salt to the organic solvent is about 1 g/1 mL to about 1 g/10 mL.

22. The method of claim 21, wherein the ratio of atorvastatin hemi-calcium salt to the organic solvent is about 1 g/5 mL to about 1 g/10 mL.

23. The method of claim 22, wherein the organic solvent is ethyl acetate, and the ratio of atorvastatin hemi-calcium salt to the organic solvent is about 1 g/5 mL.

24. The method of claim 22, wherein the organic solvent is acetone, and the ratio of atorvastatin hemi-calcium salt to the organic solvent is about 1 g/10 mL.

25. The method of claim 21, wherein the organic solvent comprises a mixture of an ester and a hydroxylic solvent, and the ratio of ester to hydroxylic solvent in the mixture is about 1:1 (on a volume/volume basis).

26. The method of claim 21, wherein the organic solvent comprises a mixture of a ketone and a hydroxylic solvent, and the ratio of ketone to hydroxylic solvent in the mixture is about 1:1 (on a volume/volume basis).

27. The method of any one of claims 1, 8 and 13, wherein the solvent is removed by spray drying.

28. The method of any one of claims 1, 8 and 13, wherein the solvent is removed by thin film evaporation.

29. The method of any one of claims 1, 8 and 13, wherein the solvent is removed by rapid vacuum evaporation.

30. The method of claim 29, wherein vacuum evaporation is performed at a pressure in the range of from about 3 Torr to about 250 Torr.

31. The method of claim 29, wherein vacuum evaporation is performed at a temperature in the range of from about 20° C. to about 60° C.

32. Amorphous atorvastatin hemi-calcium salt made by the process of any one of claims 1, 8 and 13.

33. A pharmaceutical composition comprising an amorphous atorvastatin hemi-calcium salt of claim 32, or solvate thereof.

34. A method for treating hypertension comprising the step of administering a pharmaceutical composition of claim 33.
